(19) United States
(12) Patent Application Publication
(10) Pub. No.: US 2022/0068622 A1
(43) Pub. Date: Mar. 3, 2022

(54) DISPLAY-PROCESSING DEVICE FOR MASS SPECTROMETRY DATA

(71) Applicant: SHIMADZU CORPORATION, Kyoto-shi, (JP)

(72) Inventors: Shinichi IWAMOTO, Kyoto-shi (JP); Kanae TERAMOTO, Kyoto-shi (JP)

(73) Assignee: SHIMADZU CORPORATION, Kyoto-shi, (JP)

(21) Appl. No.: 17/399,135

(22) Filed: Aug. 11, 2021

(30) Foreign Application Priority Data
Sep. 3, 2020 (JP) 2020-148365

(51) Int. Cl. H01J 49/00; G16C 20/20; G01N 33/68

(52) U.S. Cl. CPC H01J 49/0036; G01N 33/6848; G16C 20/20

(57) ABSTRACT

Provided is a display-processing device for mass spectrometry data capable of presenting a mass spectrum of a test microorganism and existing genome-related information so that the relationship between the two kinds of information can be easily understood. In the device, a spectrum acquirer (41) acquires a mass spectrum (80) of a test microorganism. A genome-related information acquirer (42) acquires genome-related information of a known microorganism which is identical or related to the test microorganism, based on the mass spectrum. A correspondence relationship determiner (43) determines a correspondence relationship between peaks on the mass spectrum and proteins expressed in the known microorganism. A display controller (45) displays, on a display device, identifiers (81) and a genome map (70) along with the mass spectrum, each identifier indicating what protein corresponds to a given peak, and the genome map showing the location of the gene encoding each protein on the genome.

### TECHNICAL FIELD

The present invention relates to a display-processing device for mass spectrometry data.

### BACKGROUND ART

In recent years, a technique for identifying microorganisms by mass spectrometry has been developed. In this technique, a liquid sample, such as a solution containing proteins extracted from a test microorganism or a suspension of a test microorganism, is initially analyzed with a mass spectrometer which employs a soft ionization method, such as MALDI (matrix assisted laser desorption/ionization). A “soft” ionization method is a type of ionization method which barely causes the fragmentation of high-molecular compounds. The obtained mass spectrum is subsequently compared with mass spectra of known microorganisms to identify the genus, species or strain of the test microorganism. Such a technique is generally called “fingerprinting” since it uses a mass-spectral pattern as a piece of information that is specific to each microorganism (i.e., a fingerprint).

The fingerprinting method has a problem in terms of the rationale for and reliability of the identification since the method does not determine the kind of protein from which each individual peak on a mass spectrum has originated. A technique has been developed for solving this problem, which utilizes the fact that approximately one half of the peaks obtained by a mass spectrometric analysis of a microorganism body originate from ribosomal proteins. According to the technique, the mass-to-charge ratio of a peak obtained by a mass spectrometric analysis is related to a calculated mass estimated from an amino-acid sequence determined by translating the base sequence information of a ribosomal protein gene, to determine the kind of protein that should be assigned to the peak concerned (for example, see Patent Literature 1). This technique enables a rational, reliable identification of microorganisms by mass spectrometry.

### CITATION LIST

### Patent Literature

Patent Literature 1: JP 2007-316063 A

### SUMMARY OF INVENTION

### Technical Problem

Determining the kind of protein that should be assigned to a mass spectrum peak requires genome information or protein information of various microorganisms. The advancement in genomic analysis of microorganisms in recent years has made it possible to easily obtain various kinds of information concerning a microorganism, such as the genome sequence, location of each gene on the genome sequence, base sequence of each gene, name of the protein encoded by each gene, and amino-acid sequence of each protein, once the species of microorganism (or other related information) is known. Those pieces of information are hereinafter called “genome-related information”.

A problem of the conventional microorganic analysis using mass spectrometry is that it is difficult for an individual in charge of the analysis to intuitively understand the relationship between a mass spectrum acquired by a mass spectrometric analysis of a test microorganism and the aforementioned kinds of existing genome-related information.

The present invention has been developed in view of the previously described point. Its objective is to present a mass spectrum of a test microorganism and existing genome-related information so that an individual in charge of the analysis can easily understand the relationship between the two kinds of information.

### Solution to Problem

A display-processing device for mass spectrometry data according to the present invention developed for solving the previously described problem is a display-processing device for mass spectrometry data configured to display mass spectrometry data on a screen of a display device, including:

a spectrum acquirer configured to acquire a mass spectrum obtained by a mass spectrometric analysis of a test microorganism;

a genome-related information acquirer configured to acquire genome-related information which includes information concerning a plurality of proteins encoded by a genome of a known microorganism which is supposed to be identical or related to the test microorganism based on the mass spectrum and information indicating the locations of a plurality of genes which respectively encode the plurality of proteins on the genome;

a correspondence relationship determiner configured to determine a correspondence relationship between a plurality of peaks on the mass spectrum and the plurality of proteins, based on the mass spectrum and the genome-related information; and

a display controller configured to display an identifier and a genome map along with the mass spectrum on the screen, where the identifier is given to at least one of the plurality of peaks and represents the correspondence relationship between the peak concerned and one of the plurality of proteins determined by the correspondence relationship determiner, while the genome map is created based on the genome-related information and shows the locations of the plurality of genes on the genome.

### Advantageous Effects of Invention

The display-processing device for mass spectrometry data according to the present invention can present a mass spectrum of a test microorganism and existing genome-related information so that an individual in charge of the analysis can easily understand the relationship between the two kinds of information.

### BRIEF DESCRIPTION OF DRAWINGS

FIG. 1 is a schematic configuration diagram of a mass spectrometry system according to one embodiment of the present invention.

FIG. 2 is a flowchart showing the procedure of the processing by the mass spectrometry system according to the embodiment.

FIG. 3 shows one example of the screen display in the embodiment.

FIG. 4 shows one example of the screen display after the selection of a peak by a user in the embodiment.

### DESCRIPTION OF EMBODIMENTS

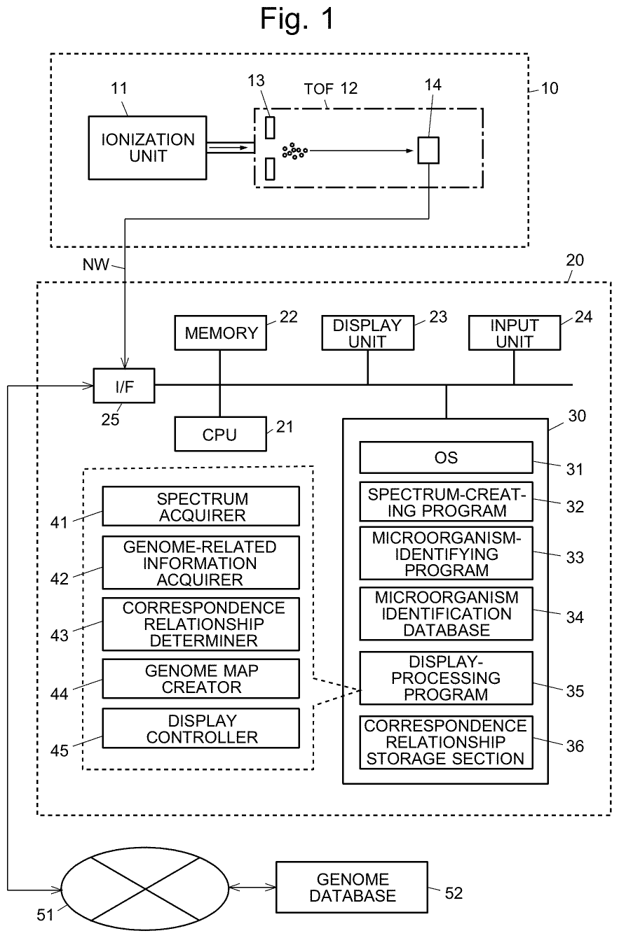

A mode for carrying out the present invention is hereinafter described with reference to the drawings. FIG. 1 is a schematic configuration diagram of a mass spectrometry system according to the present embodiment. The present mass spectrometry system includes a mass spectrometry unit 10 and an analyzing unit 20 (which is one form of the display-processing device for mass spectrometry data according to the present invention).

The mass spectrometry unit 10 includes an ionization unit 11 configured to ionize molecules or atoms in a sample by matrix assisted laser desorption/ionization (MALDI) and a time-of-flight mass separator (TOF) 12 configured to separate various ions, ejected from the ionization unit 11, according to their mass-to-charge ratios. The TOF 12 includes an extraction electrode 13 configured to extract ions from the ionization unit 11 and guide them into an ion flight space within the TOF 12, and a detector 14 configured to detect ions which have been mass-separated within the ion flight space. It should be noted that the mass spectrometry unit 10 is not limited to this configuration; it may be changed or modified in various forms.

The analyzing unit 20 is actually a workstation, personal computer or other types of computers, in which a central processing unit (CPU) 21, memory 22, display unit 23 (e.g., a liquid crystal display), input unit 24 (e.g., a keyboard and mouse), and storage unit 30 consisting of a large-capacity storage (e.g., a hard disk drive or solid state drive) are connected to each other. Stored in the storage unit 30 are an operating system (OS) 31, spectrum-creating program 32, microorganism-identifying program 33 and display-processing program 35 (which is one form of the program according to the present invention). Additionally, a microorganism identification database 34 is stored in the storage unit 30, and a correspondence relationship storage section 36 is also provided. The analyzing unit 20 further includes an interface (I/F) 25 for controlling a direct connection to an external device as well as a connection with an external device through a local area network (LAN) or other types of networks (e.g., the Internet) . Through this interface 25, the analyzing unit 20 is connected with the mass spectrometry unit 10 and a genome database 52 via a network cable NW (or wireless LAN) or the Internet 51.

In FIG. 1, a spectrum acquirer 41, genome-related information acquirer 42, correspondence relationship determiner 43, genome map creator 44 and display controller 45 are shown, being linked to the display-processing program 35. Each of those components is basically a functional means implemented at the software level by the CPU 21 executing the display-processing program 35. The display-processing program 35 does not always need to be an independent program. There is no specific limitation on its form; for example, it may be a built-in function of the microorganism-identifying program 33 or that of a program for controlling the mass spectrometry unit 10. As the microorganism-identifying program 33, for example, a program configured to identify microorganisms by a conventional fingerprinting method may be used.

In the configuration of FIG. 1, the spectrum-creating program 32, microorganism-identifying program 33, display-processing program 35, microorganism identification database 34 and correspondence relationship storage section 36 are installed on a terminal device to be operated by users. Those components, except for the display-processing program 35, may be partially or entirely installed on a separate device connected with the aforementioned terminal device via a computer network, with the separate device configured to perform the processing by those programs and/or access to the database according to commands from the terminal device. Furthermore, as opposed to FIG. 1 in which the genome database 52 is connected with the user-operated terminal device via the Internet 51, the genome database 52 may be provided in another computer located in the same facility to which the user-operated terminal device also belongs, or it may also be provided in the storage section 30 within the user-operated terminal device.

The microorganism identification database 34 holds mass lists related to a plurality of known microorganisms. A mass list is a list of the mass-to-charge ratios (m/z) of ions to be detected in a mass spectrometric analysis of the body of each known microorganism. Along with the m/z values, the list additionally includes at least the information of the classifications (e.g., family, genus, species or strain) to which the known microorganism belongs (classification information). Those mass lists can be prepared based on actual measurement data obtained beforehand by actually performing mass spectrometric analyses of various kinds of known microorganisms using the same method for ionization and mass separation as used in the mass spectrometry unit 10. When the mass lists are to be prepared from the actual measurement data, the peaks which appear within a predetermined m/z range are initially extracted from mass spectra obtained as the actual measurement data. Peaks which mainly originate from proteins can be extracted by setting the aforementioned mass-to-charge-ratio range at approximately 2000-35000, while unwanted peaks (noise) can be excluded by extracting each peak whose height (relative intensity) is equal to or higher than a predetermined threshold. Since ribosomal proteins are abundantly expressed within cells, a mass list in which most of the m/z values are of ribosomal-protein origin can be obtained by appropriately setting the aforementioned threshold. A list of the mass-to-charge ratios (m/z) and peak intensities of the peaks extracted in the previously described manner is created for each known microorganism and recorded in the microorganism identification database 34, with the aforementioned classification information and other related information added to the list. In order to reduce the variation in genetic expression due to the culture conditions, the known microorganisms to be used for collecting the actual measurement data should preferably be cultured under previously normalized conditions.

The genome database 52 holds a large number of pieces of genome-related information for each of a large number of known microorganisms. For example, the genome-related information includes the genome sequence, location of each gene on the genome sequence, base sequence of each gene, name of the protein encoded by each gene, and amino-acid sequence of each protein. Those items of genome-related information are stored in the database and related to an identifier of the known microorganism (e.g., registration number of the microorganism), name of the microorganism (e.g., genus name, species name or strain name) and other related information. For example, public databases offered by international organizations can be used as the genome database 52, such as GenBank, EMBL or DDBJ.

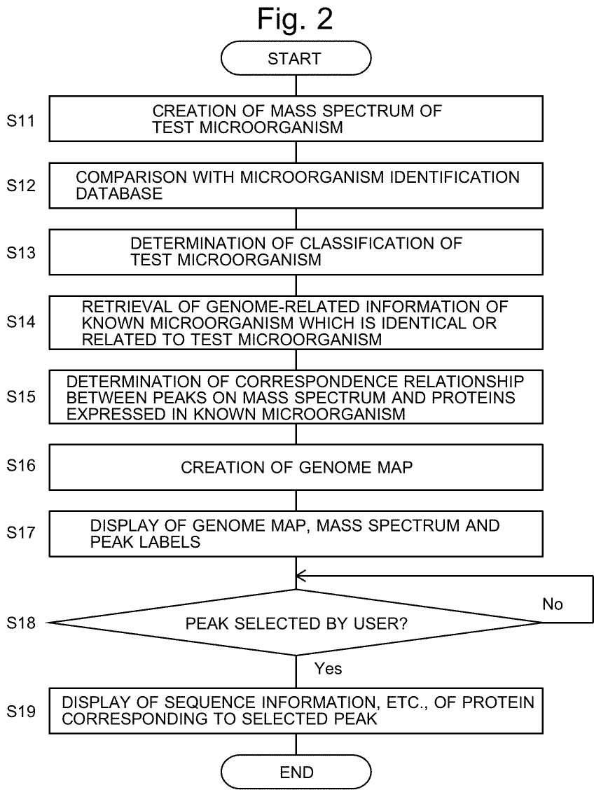

A procedure for analyzing a microorganism and displaying mass spectrometry data using the mass spectrometry system according to the present embodiment is hereinafter described with reference to the flowchart in FIG. 2.

Initially, the user prepares a sample containing the constituents of a test microorganism, sets the sample in the ionization unit 11 of the mass spectrometry unit 10, and operates the same unit to perform the mass spectrometric analysis. The sample may be an extract from the body of a test microorganism, or cell constituents (e.g., ribosomal proteins) collected from the microorganism-body extract and purified. A microorganism body or cell suspension in their original form may also be used.

When an analysis of the test sample by the mass spectrometry unit 10 is initiated, the spectrum-creating program 32 in the analyzing unit 20 receives detection signals from the detector 14 of the mass spectrometry unit 10 via the interface 25 and creates a mass spectrum for the test microorganism based on the detection signals (Step 11).

Next, the microorganism-identifying program 33 compares the mass spectrum of the test microorganism created in Step S11 with the mass lists of known microorganisms recorded in the microorganism identification database 34, and extracts a mass list having a similar m/z pattern to that of the mass spectrum of the test microorganism, such as a mass list including a considerable number of peaks whose m/z values coincide with those of the mass spectrum of the test microorganism within a predetermined margin of error (Step 12).

The microorganism-identifying program 33 subsequently refers to the microorganism identification database 34 for the classification information related to the mass list extracted in Step 12, to determine the classification (e.g., species or genus) to which the known microorganism corresponding to the mass list belongs (Step 13).

In the case where the classification of the test microorganism has been previously determined by another method, the analysis can bypass the processing by the microorganism-identifying program 33 (i.e., Steps S12 and S13) and directly proceeds to the following processing by the display-processing program 35 (i.e., Steps S14-S19).

Subsequently, the spectrum acquirer 41 in the display-processing program 35 obtains the mass spectrum of the test microorganism created in Step 11.

Next, the genome-related information acquirer 42 accesses the genome database 52 through the interface 25 and the internet 51 to retrieve the genome-related information of a known microorganism corresponding to the classification determined in Step S13, i.e., a known microorganism which is supposed to be identical or related to the test microorganism (Step S14). Specifically, for example, if the species to which the test microorganism belongs has been determined in Step S13, the genome-related information acquirer 42 searches the genome database 52, including the species name in the query, to retrieve the genome-related information of a known microorganism belonging to the species concerned.

If there are a plurality of microorganic species or microorganic strains which belong to the classification determined in Step S13 and have their genome-related information registered in the genome database 52, the genome-related information acquirer 42 retrieves genome-related information related to the type species or type strain of the plurality of microorganic species or microorganic strains. If a piece of information representing the reliability of the genome-related information related to each known microorganism is registered in the genome database, the genome-related information acquirer 42 may retrieve the most reliable information from the genome-related information related to the plurality of microorganic species or microorganic strains. For example, some of the public databases mentioned earlier contain status information which represents the progress of the genome analysis of each microorganic strain, such as “Finished”, “Permanent draft” or “Draft”. In that case, the genome information with the “Finished” status is most reliable, followed by “Permanent draft” and “Draft” in the mentioned order. If there are two or more microorganic species or microorganic strains which are comparable to each other in terms of the reliability of the genome-related information, the genome-related information acquirer 42 may retrieve the genome-related information related to the type species or type strain of those species or strains.

In the present description, it is assumed that the genome-related information acquirer 42 automatically searches the genome database 52 and retrieves appropriate genome-related information in Step S14. As another possibility, the user may perform predetermined operations using the input unit 24 to conduct a search of the genome data base 52, including the classification name determined in Step S13 in the query, and manually select a known microorganism from the search result. In that case, the genome-related information acquirer 42 retrieves the genome-related information related to the selected microorganism from the genome database 52.

Although there is only one genome database 52 shown in FIG. 1, the genome-related information acquirer 42 in the present embodiment may be configured to retrieve the aforementioned types of genome-related information from a plurality of independent genome databases (for example, databases respectively offered by different organizations).

Based on the mass spectrum created in Step S11 and the genome-related information retrieved in Step S14, the correspondence relationship determiner 43 subsequently determines the correspondence relationship between the peaks on the mass spectrum and the proteins which are known (or supposed) to be expressed in the known microorganism (Step S15). A specific procedure is as follows: Initially, the correspondence relationship determiner 43 extracts the amino-acid sequences of predetermined proteins from the genome-related information retrieved in Step S14. The “predetermined proteins” may be all proteins registered for the known microorganism in the genome database 52 or some of those proteins previously specified by the user (e.g., some or all of the ribosomal proteins). Subsequently, the correspondence relationship determiner 43 calculates the molecular weights of the predetermined proteins from their respective amino-acid sequences, and converts the calculated molecular weights into theoretical m/z values of the predetermined proteins. The “theoretical m/z value” of a protein is the m/z value of an ion which is expected to be detected by a mass spectrometric analysis of that protein. It is commonly known that an molecular-related ion, such as [M+H]⁺ (where M is the molecule and H is the hydrogen atom), [M−H]⁻ or [M+Na]⁺ (where Na is the sodium atom), is mainly detected when a biological sample is analyzed by mass spectrometry in which the sample is ionized by MALDI. Therefore, provided that the mass spectrometric conditions are fixed, it is easy to convert the calculated molecular weight of each protein into the theoretical m/z value. If the calculated molecular weight of a protein which is known (or supposed) to be expressed in the known microorganism is contained in the genome database 52, it may be used for the calculation of the theoretical m/z value. Subsequently, for each of the predetermined proteins, the correspondence relationship determiner 43 searches the mass spectrum of the test sample for a peak which falls within a predetermined margin of error from its theoretical m/z value determined in the previously described manner. A protein for which a matching peak has been found is considered to be the protein corresponding to that peak. Accordingly, the correspondence relationship determiner 43 records the relationship between the protein and the peak in the correspondence relationship storage section 36.

Subsequently, the genome map creator 44 creates a genome map which shows the location of each gene on the genome sequence of the known microorganism, based on the genome-related information retrieved in Step S14 (Step S16).

Next, the mass spectrum 80 created in Step S11, peak labels 81 showing the correspondence relationship determined in Step S14 (those labels correspond to the identifier in the present invention), and genome map 70 created in Step S16 are displayed on the screen of the display unit 23 under the control of the display controller 45 (Step S17).

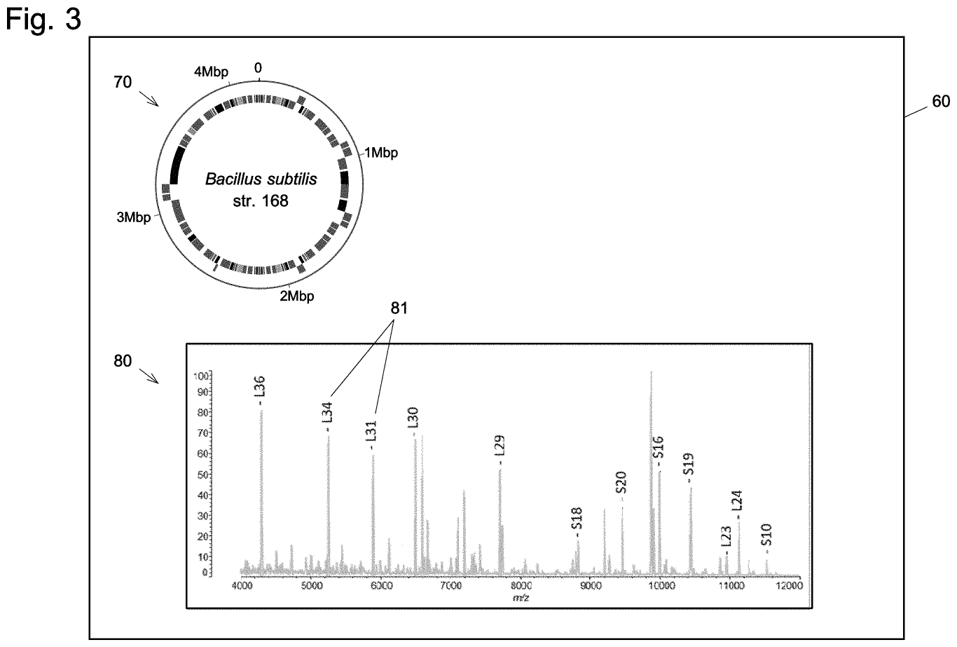

One example of the screen display in this stage is shown in FIG. 3. The genome map 70 is shown in the upper portion of the display screen 60, while the mass spectrum 80 of the test microorganism is shown in the lower portion of the display screen 60.

Furthermore, among the peaks on the mass spectrum 80, each peak for which the corresponding protein has been identified in Step S15 is denoted by the peak label 81 which shows the name of the protein corresponding to the peak. For example, the peak label 81 having the character string “L36” in FIG. 3 means that the peak corresponds to “ribosomal protein L36”.

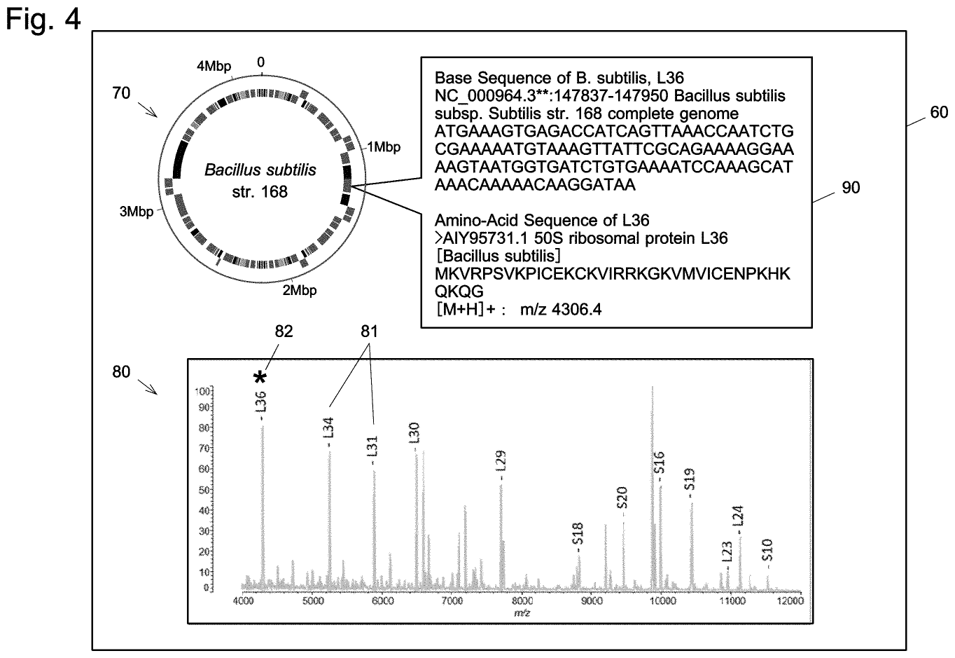

The display screen 60 shown on the display unit 23 is configured to allow the user to select one of the peaks on the mass spectrum 80 by means of the input unit 24. When a peak is selected on the display screen 60 (“Yes” in Step S18), the peak (which is hereinafter called the “selected peak”) is highlighted on the display screen 60 as shown in FIG. 4 (by a mark 82 displayed near the selected peak). Additionally, if a protein corresponding to the selected peak has already been identified in Step S15, a protein-information display box 90 which shows information concerning the protein corresponding to the selected peak (this protein is hereinafter called the “selected protein”) is displayed in the upper-right portion of the display screen 60 (Step S19). The selection of a peak by the user is made, for example, in such a manner that the user clicks on a desired peak or peak label 81 on the display screen 60. The combination of the display controller 45 and the input unit 24 in the present embodiment corresponds to the peak selection receiver in the present invention.

In FIG. 4, as one example of the highlighting, the mark 82 which denotes the selected peak is shown near the peak concerned. The form of the highlighting is not limited to this type. For example, the selected peak may be given a different color or width from the other peaks, or the peak label assigned to the selected peak may be shown in a different color or font from the other peak labels. In addition to the highlighting of the selected peak, the location of the gene which encodes the selected protein on the genome map 70 may also be highlighted.

The protein-information display box 90 is shaped like a speech balloon extending from the location of the gene which encodes the selected protein on the genome map 70. The protein-information display box 90 shows various pieces of information related to the selected protein, including the name of the selected protein, base sequence of the gene which encodes the selected protein, identification number of the same gene on the genome database 52, amino-acid sequence and theoretical m/z value of the selected protein, as well as identification number of the selected protein on the genome database 52.

Thus, the mass spectrometry system according to the present embodiment displays a mass spectrum of a test microorganism and existing genome-related information so that the user can easily understand the relationship between the two kinds of information. Therefore, for example, even a microorganism researcher or other individuals who are inexperienced in an analysis of mass spectra can easily understand the result of a mass spectrometric analysis of a test microorganism.

### [Various Modes of Invention]

A person skilled in the art can understand that the previously described illustrative embodiment is a specific example of the following modes of the present invention.

(Clause 1) A display-processing device for mass spectrometry data according to one mode of the present invention is a display-processing device for mass spectrometry data configured to display mass spectrometry data on a screen of a display device, including:

a spectrum acquirer configured to acquire a mass spectrum obtained by a mass spectrometric analysis of a test microorganism;

a genome-related information acquirer configured to acquire genome-related information which includes information concerning a plurality of proteins encoded by a genome of a known microorganism which is supposed to be identical or related to the test microorganism based on the mass spectrum and information indicating the locations of a plurality of genes which respectively encode the plurality of proteins on the genome;

a correspondence relationship determiner configured to determine a correspondence relationship between a plurality of peaks on the mass spectrum and the plurality of proteins, based on the mass spectrum and the genome-related information; and

a display controller configured to display an identifier and a genome map along with the mass spectrum on the screen, where the identifier is given to at least one of the plurality of peaks and represents the correspondence relationship between the peak concerned and one of the plurality of proteins determined by the correspondence relationship determiner, while the genome map is created based on the genome-related information and shows the locations of the plurality of genes on the genome.

The display-processing device for mass spectrometry data described in Clause 1 allows the user to instantaneously understand the kind of protein which each peak on the mass spectrum corresponds to, as well as the location at which the gene which encodes the protein exists on the genome.

(Clause 2) In the display-processing device for mass spectrometry data described in Clause 1, the display-processing device for mass spectrometry data according to another mode of the present invention further includes:

a peak selection receiver configured to allow a user to select one peak from the plurality of peak on the mass spectrum displayed on the screen, where:

the display controller is configured to highlight, on the genome map, the location of a gene which encodes a protein corresponding to the peak selected through the peak selection receiver among the plurality of proteins.

The display-processing device for mass spectrometry data described in Clause 2 creates a screen display on which the user the location of the gene corresponding to a desired peak on the genome can intuitively understand. The user only needs to select the desired peak.

(Clause 3) In the display-processing device for mass spectrometry data described in Clause 1, the display-processing device for mass spectrometry data according to another mode of the present invention further includes:

a peak selection receiver configured to allow a user to select one peak from the plurality of peak on the mass spectrum displayed on the screen, where:

the genome-related information further includes information concerning the amino-acid sequences of the plurality of proteins or the base sequences of the genes which respectively encode the proteins; and

the display controller is further configured to display, on the screen, the amino-acid sequence of a protein corresponding to the peak selected through the peak selection receiver among the plurality of proteins, or the base sequence of the gene which encodes the protein.

The display-processing device for mass spectrometry data described in Clause 3 allows the user to easily refer to the amino-acid sequence of a protein or base sequence of a gene corresponding to a desired peak. The user only needs to select the desired peak.

(Clause 4) A program according to another mode of the present invention is a program configured to make a computer function as the display-processing device for mass spectrometry data described in one of Clauses 1-3.

### REFERENCE SIGNS LIST

10 . . . Mass Spectrometry Unit
20 . . . Analyzing Unit
30 . . . Storage Section
32 . . . Spectrum-Creating Program
33 . . . Microorganism-Identifying Program
34 . . . Microorganism Identification Database
35 . . . Display-Processing Program
36 . . . Correspondence Relationship Storage Section
41 . . . Spectrum Acquirer
42 . . . Genome-Related Information Acquirer
43 . . . Correspondence Relationship Determiner
44 . . . Genome Map Creator
45 . . . Display Controller
52 . . . Genome Database
60 . . . Display Screen
70 . . . Genome Map
80 . . . Mass Spectrum
81 . . . Peak Label
82 . . . Mark
90 . . . Protein-Information Display Box

1. A display-processing device for mass spectrometry data configured to display mass spectrometry data on a screen of a display device, comprising:
a spectrum acquirer configured to acquire a mass spectrum obtained by a mass spectrometric analysis of a test microorganism;
a genome-related information acquirer configured to acquire genome-related information which includes information concerning a plurality of proteins encoded by a genome of a known microorganism which is supposed to be identical or related to the test microorganism based on the mass spectrum and information indicating locations of a plurality of genes which respectively encode the plurality of proteins on the genome;
a correspondence relationship determiner configured to determine a correspondence relationship between a plurality of peaks on the mass spectrum and the plurality of proteins, based on the mass spectrum and the genome-related information; and
a display controller configured to display an identifier and a genome map along with the mass spectrum on the screen, where the identifier is given to at least one of the plurality of peaks and represents the correspondence relationship between the peak concerned and one of the plurality of proteins determined by the correspondence relationship determiner, while the genome map is created based on the genome-related information and shows the locations of the plurality of genes on the genome.

2. The display-processing device for mass spectrometry data according to claim 1, further comprising:
a peak selection receiver configured to allow a user to select one peak from the plurality of peak on the mass spectrum displayed on the screen, where:
the display controller is configured to highlight, on the genome map, the location of a gene which encodes a protein corresponding to the peak selected through the peak selection receiver among the plurality of proteins.

3. The display-processing device for mass spectrometry data according to claim 1, further comprising:
a peak selection receiver configured to allow a user to select one peak from the plurality of peak on the mass spectrum displayed on the screen, where:
the genome-related information further includes information concerning amino-acid sequences of the plurality of proteins or base sequences of the genes which respectively encode the proteins; and
the display controller is further configured to display, on the screen, the amino-acid sequence of a protein corresponding to the peak selected through the peak selection receiver among the plurality of proteins, or the base sequence of the gene which encodes the protein.

4. A non-transitory computer readable medium recording a program configured to make a computer function as the display-processing device for mass spectrometry data according to claim 1.
